(19) United States
(12) Patent Application Publication
(10) Pub. No.: US 2004/0122438 A1
(43) Pub. Date: Jun. 24, 2004

(54) Flex-tight interlocking connection tubing for delivery of bone cements/biomaterials for vertebroplasty

(75) Inventor: Robert M. Abrams, Los Gatos, CA (US)

Correspondence Address: BINGHAM, MCCUTCHEN LLP, THREE EMBARCADERO, SUITE 1800, SAN FRANCISCO, CA, 94111-4067, US

(73) Assignee: Boston Scientific Corporation

(21) Appl. No.: 10/329,150

(22) Filed: Dec. 23, 2002

(51) Int. Cl. A61B 17/56

(52) U.S. Cl. 606/93

(57) ABSTRACT

An apparatus for delivering compounds into a vertebra includes a plurality of sequential tubes connected to one another by pivotable joints, the tubes including a first end, a second end, and a lumen extending therebetween. Each pivotable joint between adjacent tubes includes a locking mechanism, e.g., a clamp or locking nut, for securing the adjacent tubes relative to one another. During use, a needle is inserted into the vertebra, one end of the tubes is connected to the needle and a syringe or other delivery device is connected to the other end. The joints between the tubes are locked, thereby substantially securing the tubes from pivotal movement relative to one another, and the compound, e.g., bone cement and/or biomaterials, is injected through the tubes and out the needle into the tissue structure.

### FIELD OF THE INVENTION

The present invention relates generally to apparatus and methods for delivering compounds into a body, and more particularly to apparatus and methods for delivering bone cements, biomaterials, and/or other flowable compounds into vertebrae, e.g., during a vertebroplasty procedure.

### BACKGROUND

Vertebroplasty is a procedure during which bone cement, biomaterials, and/or other compounds are delivered into a vertebra.

A syringe or other delivery device is generally provided within which the bone cement to be delivered is stored. For example, the delivery device may include a barrel or housing including an open inlet end and an exit end with a narrow outlet. A plunger or threaded driver may be advanced into the inlet end to force bone cement within the barrel out the outlet in the exit end.

A needle may be inserted percutaneously through the cutaneous layers of tissue above a hard tissue structure being treated and into the hard tissue structure. For example, the hard tissue structure may be a vertebra, and the needle may penetrate through cortical bone and into the cancellous bone within the vertebra. Alternatively, the hard tissue structure may be at least partially exposed using conventional surgical procedures before inserting the needle.

A semi-rigid or flexible tube, e.g., twenty to fifty centimeters long, may be connected between the proximal end of the needle and the outlet of the delivery device to deliver the bone cement via the tube into the hard tissue structure. The tube may be bent slightly during the procedure, e.g., to keep the user's hands and/or the delivery device out of the field of an imaging device, such as a fluoroscope, that may be used to monitor the procedure.

Because of the high viscosity of the bone cement, high pressures are required to inject the bone cement from the delivery device through the tube and needle into the hard tissue structure. For example, pressures of up to one to three thousand pounds per square inch (1,000-3,000 psi) may be required to inject bone cement from the delivery device, through the tube and needle, and into the hard tissue structure. This requires the user to apply substantial force, while simultaneously supporting the weight of the delivery device and its contents. This may cause fatigue of the user and/or undesired movement of the delivery device during the procedure.

Accordingly, apparatus and methods for delivering bone cement or other compounds into vertebrae would be useful.

### SUMMARY OF THE INVENTION

The present invention is directed to apparatus and methods for delivering fluids into a body, and more particularly to apparatus and methods for delivering bone cements and/or biomaterials into vertebrae, e.g., during a vertebroplasty procedure.

In accordance with one aspect of the present invention, an apparatus is provided for delivering a compound into a tissue structure. The apparatus includes a first tubular member including first and second ends and a lumen extending therebetween, and a second tubular member including first and second ends and a lumen extending therebetween. The first end of the second tubular member may be pivotally coupled to the second end of the first tubular member such that the lumens of the first and second tubular members communicate with one another.

In a preferred embodiment, the mating ends of the first and second tubular members may include a ball and socket, providing a ball joint. The joint may have at least one degree of freedom, and preferably two degrees of freedom (pivoting in two orthogonal planes).

Optionally, the apparatus may include a third or more additional tubular members, each of which may be pivotally coupled to an adjacent tubular member. Each tubular member may be substantially rigid and/or substantially straight or curved (radiused).

A locking mechanism may be carried by at least one of the first and second tubular members for selectively securing the first and second tubular members from pivotal movement relative to one another. The locking mechanism may be a locking nut surrounding the joint, the locking nut being securable against the joint to prevent pivotal movement of the first and second tubular members relative to one another.

Alternatively, the locking mechanism may be a clamp that may be secured against the joint to prevent pivotal movement of the first and second tubular members relative to one another. For example, the clamp may include a “C” shaped body that may be received around the joint and a nut connected to ends of the “C” shaped body. The “C” shaped body may compress the joint when the nut is tightened onto the ends to secure the first and second tubular members from pivotal movement relative to one another.

The apparatus may include a needle or other cannula communicating with the second end of the second tubular member. The cannula may include an outlet, e.g., in a tissue-piercing or blunt end, communicating with the lumen of the second tubular member for delivering a compound passing through the lumens out the outlet. Optionally, the apparatus may include a source containing a compound coupled to the first end of the first tubular member, e.g., a syringe or other injector including bone cement, biomaterials, and/or other flowable materials therein.

In accordance with another aspect of the present invention, a system for delivering compounds into a tissue structure, e.g., a vertebra or other hard tissue structure. The system may include a plurality of tubes connected sequentially to one another by pivotable joints, the tubes including a first end and a second end and a lumen extending therebetween. A locking mechanism may be provided at each pivotable joint between adjacent tubes for securing the adjacent tubes relative to one another. A source of flowable compound for treating a tissue structure may be coupled to the first end of the plurality of tubes and/or a needle may be coupled to the second end of the plurality of tubes that includes an outlet communicating with the lumen of the tubes for delivering the compound out the outlet from the source of flowable material via the lumen of the tubes.

In accordance with yet another aspect of the present invention, a method is provided for delivering a compound into a tissue structure using a plurality of tubes connected sequentially to one another by pivotable joints. A cannula may be inserted into the tissue structure to be treated. For example, the tissue structure may be a hard tissue structure, e.g., a bone. Preferably, tissue structure is a vertebra, and the cannula is a needle that may penetrate through cortical bone into cancellous bone of the vertebra as the needle is inserted into the vertebra.

One end of the tubes may be connected to the cannula either before or after the cannula is inserted into the tissue structure. A syringe or other delivery device may be connected to the other end of the tubes, either before or after connecting the needle to the one end of the tubes. The delivery device may be manipulated or otherwise disposed at a desired location relative to the cannula and/or tissue structure being treated. For example, it may be desirable to position the delivery device such that the delivery device and/or the user's hand(s) are out of the field of an imaging device, e.g., a fluoroscope, that may be used during the procedure.

Joints between the plurality of tubes may be locked, thereby substantially securing the tubes from pivotal movement relative to one another. A compound, e.g., bone cement and/or biomaterials, may be injected through the tubes and out the cannula into the tissue structure, e.g., into the cancellous bone region of a vertebra. For example, a plunger, trigger, lever, and the like on the delivery device may be manipulated to generate sufficient force needed to inject the compound within the delivery device through the tubes and the cannula into the tissue structure.

Because of the rigidity of the tubes, once the joints are locked, the tubes may at least partially support the delivery device. Thus, the user may not have to support the entire weight of the delivery device during the procedure. In addition, the tubes may prevent the delivery device from moving substantially while force is applied to inject the compound from the delivery device into the tissue structure.

Other objects and features of the present invention will become apparent from consideration of the following description taken in conjunction with the accompanying drawings.

### BRIEF DESCRIPTION OF THE DRAWINGS

FIG. 1 is a perspective view of a preferred embodiment of an apparatus for delivering flowable materials into a vertebra, in accordance with the present invention.

FIG. 2 is a perspective view of an exemplary embodiment of a tubular member including a locking nut thereon that may be included in the apparatus of FIG. 1.

FIG. 3 is a perspective view of an alternative embodiment of a tubular member including a clamping ring thereon that may be included in the apparatus of FIG. 1.

### DETAILED DESCRIPTION OF THE PREFERRED EMBODIMENTS

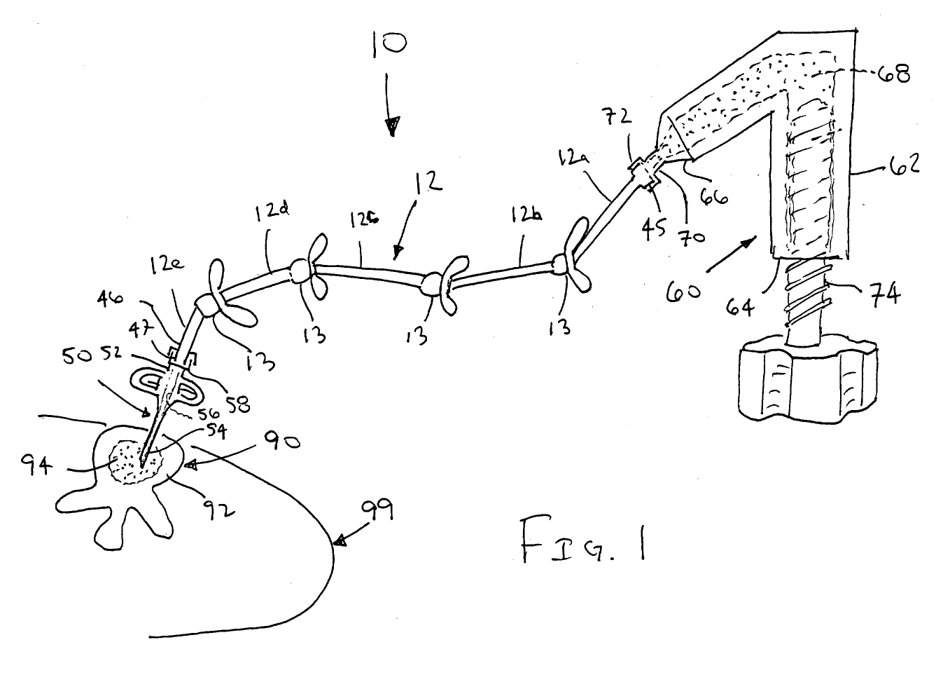

Turning to the drawings, FIG. 1 shows a first preferred embodiment of an apparatus 10 for delivering bone cement, biomaterial, and/or other compounds into a vertebra 90 or other hard tissue structure (not shown) of a patient 99. Generally, the apparatus 10 includes a plurality of tubes 12 connected sequentially to one another by pivotable joints 13, and a needle or other cannula 50. In addition, the apparatus 10 may include a delivery device 60 or other source of bone cement and/or other compound that may be coupled to one end of the plurality of tubes 12.

In the exemplary embodiment shown, five tubes 12a-12e are shown. It will be appreciated, however, that any number of tubes 12 may be provided depending upon the number of degrees of articulation and the complexity required by a given situation. Preferably, the tubes 12 are substantially rigid, although alternatively the tubes 12 may be semi-rigid or flexible. The tubes 12 may be extruded, machined, rolled, molded, or otherwise formed from materials, e.g., metals, such as Nitinol or stainless steel, or plastics that are compatible with the compounds delivered through the tubes 12 (such as PMMA monomer). In a preferred embodiment, the tubes 12 may be formed from a braided or helical coil embedded polymer, such as polyimide or PEEK. The tubes 12 may have an outer diameter between about 1.5-2.5 millimeters, a wall thickness between about 0.25-0.75 millimeter, and a length between about two hundred and five hundred millimeters.

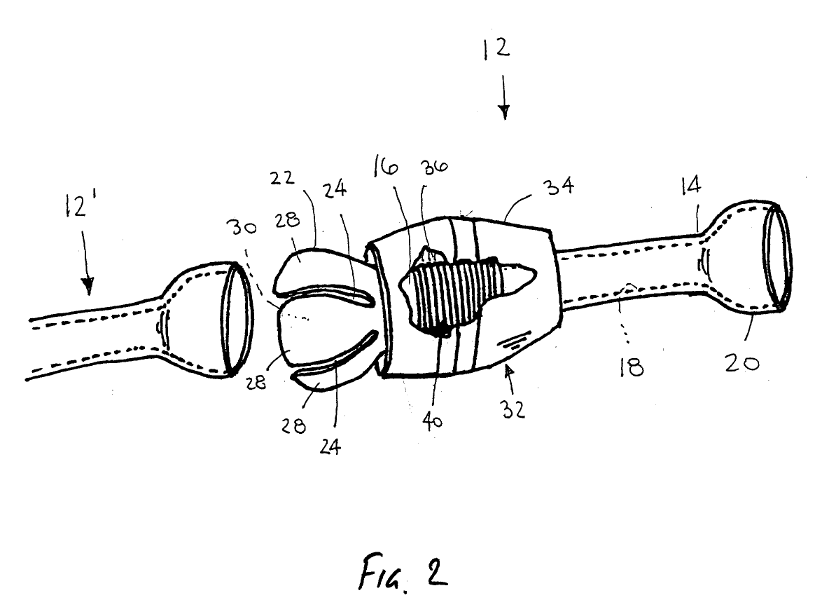

Each tube 12 includes a first end 14, a second end 16, and a lumen 18 extending therebetween. Turning to FIG. 2, for intermediate tubes, such as tubes 12b-12d of FIG. 1, the first end 14 may include a hollow ball 20, while the second end 16 may include a mating socket 22. Thus, the ball 20 from one tube 12 may be pivotally received in the mating socket 22 of an adjacent tube 12 to provide a ball joint, as explained further below. The ball 20 and/or socket 22 may include one or more seals (not shown) for providing a fluid-tight joint between the ball joint, as is well known in the art.

Preferably, at least one of the ball 20 and the socket 22 includes elements that allow the ball 20 or socket 22 to be expanded radially outwardly and/or compressed radially inwardly. For example, as shown in FIG. 2, the socket 22 may include a plurality of slots 24 extending substantially parallel to a longitudinal axis 26 of the tube 12, thereby defining a plurality of tongues 28 that define the cavity 30 within the socket 22. Thus, the socket 22 may operate as a collet, e.g., reducing the cross-section of the cavity 30 when the tongues 28 are compressed radially inwardly to capture a ball (not shown) received therein. Preferably, the cavity 30 has a partial spherical shape such that a ball from an adjacent tube (not shown) may be rotatably received within the cavity 30. More preferably, the socket 22 includes a cavity 30 defining more than half of a sphere such that a ball captured therein may not be easily removed.

Each tube 12 may include a locking mechanism thereon that may be used to compress or expand the ball 20 and/or socket 22 of the tube 12. The locking mechanism may secure a mating ball and socket, i.e., a ball joint 13 between the tube 12 and an adjacent tube (not shown), to prevent rotational movement relative to one another.

In a preferred embodiment, shown in FIG. 2, the locking mechanism may be an annular locking nut 32 that is slidably received over the tube 12. The locking nut 32 may include an annular body 34 including one or more threads 36 along its inner surface. The annular body 34 may have an outer shape to facilitate manipulating the locking nut 32, e.g., moving the locking nut 32 axially along the tube 12 and/or rotating the locking nut 32 about the tube 12. For example, the annular body 34 may have a hexagonal or other shape (not shown) along all or a portion of the outer surface for manipulating the locking but 32 either manually or using a tool. Alternatively, a plurality of wings or other elements (not shown) may extend from the annular body 34 for manipulating the locking nut 32.

The second end 16 of the tube 12 may include one or more threads 40 on its outer surface that may slidably mate with the threads 36 on the locking nut 32. Alternatively or in addition, the threads (not shown) may extend along an enlarged portion of the socket 22 relative to the wall of the tube 12. Thus, as the locking nut 32 is threaded onto the threads 40, the tongues 28 may be compressed inwardly, thereby reducing the cross-section of the cavity 30 of the socket 22.

During use, a ball of an adjacent tube (not shown) may be received in the socket 22, and the locking nut 32 may be threaded onto the socket 22 to compress the tongues 28 of the socket 22 inwardly to bear against the ball, thereby preventing the ball from being removed from the socket 22 and/or rotating within the socket 22. Thus, an adjacent tube, e.g., tube 12′ in FIG. 2, may be received in the socket 22 and pivoted relative to the tube 12, and then secured in a particular orientation by tightening the locking nut 32. To adjust the relative angular orientation of the tube 12 and the adjacent tube 12′, the locking nut 32 may be unthreaded to release the ball within the socket 22, the tube 12′ pivoted as desired, and the locking nut 32 retightened.

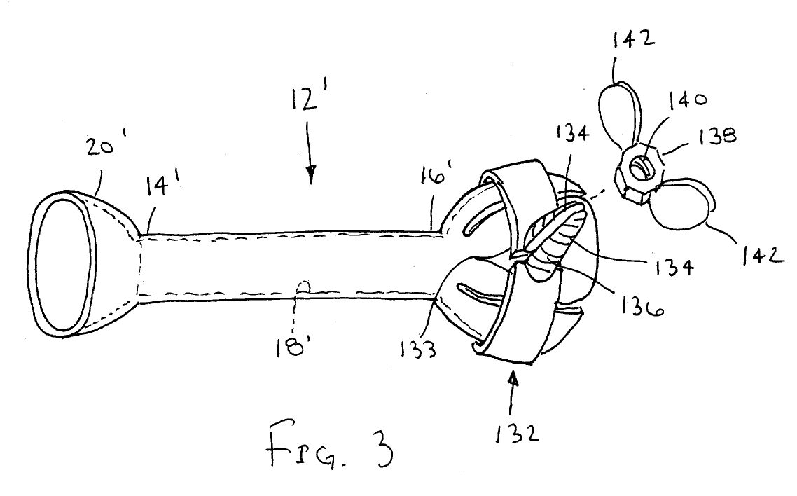

Alternatively, as shown in FIG. 3, the locking mechanism may be a clamping ring 132 that slidably fits around a tube 12.′ The clamping ring 132 may include a “C” shaped body including opposing ends 133, and a nut 138. Threaded hubs 134 may extend from the ends 133, e.g., having partial semi-cylindrical shapes such that the hubs 134 together generally define a cylindrical shaft. Preferably, the hubs 134 are tapered, e.g., such that the base is wider than the end of the hubs 134, as shown in FIG. 3B, thereby defining a frusto-conical shape. One or more threads 136 may be provided on the outer surface of the hubs 134.

The nut 138 may include one or more threads 140 along its inner surface that may mate with the threads 136 on the hubs 134 such that the nut 138 may be threaded onto the hubs 134. The nut 138 may also include a plurality of wings 142 or other elements that may facilitate rotating or otherwise manipulating the nut 138 during use. Alternatively, the nut 138 may include a hexagonal or other external shape (not shown) for manipulating the nut 138, either manually or using a tool.

The clamping ring 132 may be received around the tube 12,′ preferably around the socket 22.′ The socket 22′ may include a groove (not shown) extending about the circumference of the socket 22,′ e.g., in the outer surface of the tongues 28.′ Thus, the clamping ring 132 may be retained axially relative to the socket 22′ but may be free to rotate around the socket 22.′ Optionally, the clamping ring 132 and/or the groove may include a seal (not shown) to prevent fluid from leaking through the socket 22.′

During use, a ball of an adjacent tube (not shown) may be received in the socket 22′ and the clamping ring 132 may be tightened to secure the ball within the socket 22. Preferably, the nut 138 is threaded down onto the hubs 134 of the clamping ring 132. Because of the tapered shape of the hubs 134, as the nut 138 is threaded down, the ends 133 of the clamping ring 132 are drawn towards one another, thereby compressing the tongues 28′ of the socket 22′ inwardly around the ball. If it is desired to adjust the tube (not shown) connected to the ball, the nut 138 may be unthreaded to loosen the clamping ring 132 and release the socket 22.′ The adjacent tube may be pivoted to a desired orientation, and the nut 136 tightened back down.

Returning to FIG. 1, the end tubes 12a, 12e may include a ball or socket on one end, as described above. The other end of the end tubes 12a, 12e, however, may include a connector for mating with other components of the apparatus 10, such as the needle 50 and the delivery device 60. For example, the proximal end 44 of the tube 12a may include a male or female luer lock or other connector 45 that may engage with a complementary connector 72 on an outlet port 70 of the delivery device 60. Similarly, the distal end 46 of the tube 12e may include a luer lock or other connector 47 that may engage with a complementary connector 58 on the proximal end of the needle 50.

The needle 50 is generally of conventional design, e.g., a substantially rigid cannula or other elongate body including a proximal end 52, a distal end 54, and a lumen 56 extending therebetween. A luer lock or other connector 58 may be provided on the proximal end 52 for mating with the connector 47 on the distal end 46 of the tube 12e. Alternatively, the proximal end 52 of the needle 50 and the adjacent tube 12e may include ball and socket ends (not shown) such that the needle 50 may be pivotally coupled to the tube 12e.

The needle 50 may be substantially straight or, alternatively, may include a preset bend, e.g., between about forty five and ninety degrees (45-90°). In a further alternative, the needle 50 may include an elbow joint, e.g., of about ninety degrees (90°), with ball and socket ends (not shown), which may facilitate maneuvering the tubes 12 and/or delivery device 60 without disturbing the distal end 54 of the needle 50 (which may be positioned inside a vertebra or other bone (not shown).

The distal end 54 of the needle 50 may be beveled or otherwise sharpened such that the distal end 54 may penetrate into hard tissue, such as bone. Alternatively, the distal end 54 may be substantially blunt or rounded (not shown). The needle 50 may taper from the proximal end 52 to the distal end 54, e.g., such that the distal end 54 corresponds to a conventional needle diameter, e.g., between about eleven to thirteen gauge (11-13 GA).

The apparatus 10 may also include a delivery device 60, such as a syringe or other injector. Generally, the delivery device includes a barrel 62 including an open proximal end 64, and a closed distal end 66, thereby defining an interior space 68 within which a compound, such as bone cement and/or biomaterials, may be contained. The distal end 66 includes an outlet port 70 communicating with the interior space 68. A luer lock or other connector 72 may be provided on the outlet port 70 for cooperating with a complementary connector, such as the connector 45 on the tube 12a, as explained above.

A piston and/or plunger 74 may be inserted into the proximal end 64 of the barrel 62 for forcing a compound within the barrel 62 out through the outlet port 70. The plunger 74 may be advanced distally, thereby applying a force creating sufficient pressure to inject the compound out the outlet port 70. Alternatively, the plunger 74 and/or barrel 62 may include mating threads (not shown) such that the plunger 74 may be rotated to advance it distally into the barrel 62. Levers, triggers, or other mechanisms may be used to facilitate injecting the compound from within the barrel 62, particularly because of the relatively high pressures necessary to deliver bone cement and like compounds, as is well known in the art. Exemplary embodiments of a delivery device that may be used in conjunction with the present invention are disclosed in published PCT applications WO 02/064062 and WO 99/49819, the disclosures of which are expressly incorporated herein by reference.

The apparatus 10 may be used to deliver bone cement, biomaterials, and/or other flowable compounds into a hard tissue structure. The compound may include any known material, such as those disclosed in the PCT applications incorporated by reference above. Preferably, the tissue structure is a vertebra 90, as shown in FIG. 1, although it will be appreciated that the apparatus 10 may be used to treat other hard tissue structures as well.

The distal end 54 of the needle 50 may be inserted into the vertebra 90, e.g., until the distal end 54 penetrates the cortical bone 92 and enters the cancellous bone 94 therein. The needle 50 may be inserted percutaneously, e.g., through cutaneous tissue, fat, and/or muscle (not shown) overlying the vertebra 90. During insertion, an obturator, trocar, or other device (not shown) may be inserted into the lumen 56 of the needle 50 to prevent tissue and/or fluid, such as blood, from entering the lumen 56 and/or to facilitate penetrating tissue.

Alternatively, the vertebra 90 may be at least partially exposed before inserting the needle 50, e.g., using an open surgical procedure. For example, the tissue overlying the vertebra 90 may be surgically dissected and/or retracted to expose the vertebra 90, and the distal end 54 of the needle 50 may be inserted directly into the exposed vertebra 90. In a further alternative, a stylet and cannula (not shown) may be percutaneously inserted into the overlying tissue to access the vertebra 90. The stylet may be removed from within the cannula, and the needle 50 may be advanced through the cannula and then inserted into the vertebra 90. It will be appreciated that any known open or minimally invasive procedure may be used to place the needle 50 into the vertebra 90.

Once the distal end 54 of the needle 50 is inserted into the vertebra 90, the tubes 12 may be connected to the proximal end 52 of the needle 50. Specifically, the complementary connectors 47, 58 on the tube 12e and the proximal end 52 of the needle 50 may be mated with one another to secure the tubes 12 to the needle 50. The locking mechanisms, e.g., locking nuts 32, at the joints between adjacent tubes 12 may be loosened before or after connecting the tubes 12 to the needle 50. Thus, the tubes 12 may be moved relatively freely to accommodate connection to the needle 50 without imposing any stress on the needle 50 and/or the vertebra 90.

A source of material, e.g., the delivery device 60 may then be coupled to the proximal end 44 of the tube 12a, e.g., by connecting mating luer lock connectors 45, 72. Once the apparatus 10 is assembled, the delivery device 60 may be disposed at a desired location relative to the vertebra 90, the joints between the tubes 12 pivoting to accommodate positioning the delivery device 60. For example, it may be desirable to position the delivery device 60 out of the field of an imaging system, e.g., a fluoroscope, that may be used to monitor delivering the compound within the delivery system 60 into the vertebra 90.

Once the delivery device 60 is disposed at a desired location, the locking nuts 32 may be tightened, thereby securing the respective adjacent tubes 12 relative to one another. Once tightened, the tubes 12 may be disposed in a substantially rigid configuration, thereby preventing the delivery device 60 from moving substantially relative to the needle 50 and vertebra 90. Thus, unlike flexible tubes, which may kink if bent, the arrangement of tubes 12 may remain rigid and self-supporting. In addition, the tubes 12 may at least partially support the delivery device 60, thereby allowing a user, e.g., physician or other medical personnel, to hold the delivery device 60 without having to support its entire weight. If desired, the needle 50 and/or one or more of the tubes 12 may be supported by a frame or other structure (not shown) that may be secured to a table (also not shown) on which the patient is disposed or otherwise relative to the patient to support the delivery device 60 without imposing its weight upon the vertebra 90 being treated.

The compound within the delivery device 60, e.g., bone cement and/or biomaterials, may then be injected through the tubes 12 and needle 50 into the vertebra 90. The rigidity of the secured tubes 12 may allow the user to apply greater force to the delivery device 60 without concern that the delivery device 60 will move relative to the user and/or the patient. Once the compound is delivered, the needle 50 may be removed from the vertebra 90 and the tract or access area closed using conventional procedures.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

What is claimed is:

1. An apparatus for delivering a compound into a tissue structure, comprising:
a first tubular member comprising first and second ends and a lumen extending therebetween;
a second tubular member comprising first and second ends and a lumen extending therebetween, the first end of the second tubular member being pivotally coupled to the second end of the first tubular member such that the lumens of the first and second tubular members communicate with one another;
a locking mechanism carried by at least one of the first and second tubular members for selectively securing the first and second tubular members from pivotal movement relative to one another; and
a cannula communicating with the second end of the second tubular member and comprising an outlet communicating with the lumen of the second tubular member for delivering a compound passing through the lumens out the outlet.

2. The apparatus of claim 1, wherein the second end of the first tubular member and the first end of the second tubular member are coupled to one another by a ball joint.

3. The apparatus of claim 2, wherein the locking mechanism comprises a locking nut surrounding the ball joint, the locking nut being securable against the ball joint to prevent pivotal movement of the first and second tubular members relative to one another.

4. The apparatus of claim 2, wherein the locking mechanism comprises a clamp that may be secured against the ball joint to prevent pivotal movement of the first and second tubular members relative to one another.

5. The apparatus of claim 4, wherein the clamp comprises a “C” shaped body received around the ball joint and a nut connected to ends of the “C” shaped body, the “C” shaped body compressing the ball joint when the nut is tightened onto the ends to secure the first and second tubular members from pivotal movement relative to one another.

6. The apparatus of claim 1, wherein the first and second tubular members are coupled to one another by a joint having at least one degree of freedom.

7. The apparatus of claim 6, wherein the first and second tubular members are coupled to one another by a joint having two degrees of freedom.

8. The apparatus of claim 1, further comprising:
a third tubular member comprising first and second ends, the first end pivotally coupled to the second end of the second tubular member, the second of the third tubular member communicating with the needle; and
a locking mechanism for securing the second and third tubular members from pivotal movement relative to one another.

9. The apparatus of claim 1, wherein the first and second tubular members are substantially rigid.

10. The apparatus of claim 1, further comprising a source containing a flowable compound coupled to the first end of the first tubular member

11. An apparatus for delivering a compound into a tissue structure, comprising:
a plurality of tubes connected to one another by pivotable joints, the tubes including a first end and a second end and a lumen extending therebetween
a locking mechanism at each pivotable joint for securing the adjacent tubes relative to one another;
a source of compound for treating a tissue structure coupled to the first end of the plurality of tubes; and
a cannula coupled to the second end of the plurality of tubes and comprising an outlet communicating with the lumen of the tubes for delivering the compound out the outlet from the source of flowable material via the lumen of the tubes.

12. The apparatus of claim 11, wherein the pivotable joints comprise ball joints.

13. The apparatus of claim 12, wherein the locking mechanism comprises at least one of a locking nut that surrounds the ball joint, the locking nut being securable against the respective ball joint to prevent pivotal movement of the adjacent tubes relative to one another.

14. The apparatus of claim 12, wherein the locking mechanism comprises a clamp that may be secured against the ball joint to prevent pivotal movement of the first and second tubular members relative to one another.

15. The apparatus of claim 11, wherein the plurality of tubes are substantially rigid.

16. A method for delivering material into a tissue structure using a plurality of tubes connected sequentially to one another by pivotable joints, the method comprising:
inserting a cannula into the tissue structure to be treated;
connecting one end of the tubes to the cannula;
locking joints between the plurality of tubes, thereby substantially securing the tubes from pivotal movement relative to one another; and
injecting material through the tubes and out the cannula into the tissue structure.

17. The method of claim 16, wherein the cannula comprises a needle that is inserted percutaneously through cutaneous tissue overlying the tissue structure before being inserted into the tissue structure.

18. The method of claim 16, wherein the cannula is inserted into the tissue structure before the one end of the tubes is connected to the needle.

19. The method of claim 16, wherein the material comprises at least one of bone cement and biomaterial.

20. The method of claim 16, wherein the tissue structure comprises a hard tissue structure.

21. The method of claim 20, wherein the hard tissue structure comprises a vertebra, and wherein the cannula comprises a needle, the needling penetrating through cortical bone of the vertebra into cancellous bone of the vertebra when inserted into the vertebra.

22. The method of claim 16, wherein the material is contained in a delivery device that is connected to an end of the tubes opposite the cannula.

23. The method of claim 22, further comprising adjusting a position of the delivery device relative to the cannula before locking the joints between the plurality of tubes, the tubes being free to pivot relative to one another as the delivery device is adjusted.
